(19) United States
(12) Patent Application Publication
(10) Pub. No.: US 2024/0335424 A1
(43) Pub. Date: Oct. 10, 2024

(54) COMPOSITIONS AND METHODS FOR TREATING LIPOMA PAIN

(71) Applicant: Raziel Therapeutics Ltd., Rehovot (IL)

(72) Inventors: Alon BLOOMENFELD, Hod Hasharon (IL); Racheli GUETA, Rehovot (IL); Eran BLAUGRUND, Rehovot (IL)

(21) Appl. No.: 18/292,797

(22) PCT Filed: Jul. 27, 2022
(86) PCT No.: PCT/IB2022/000437

§ 371 Date: Jan. 26, 2024

Related U.S. Application Data
(60) Provisional application No. 63/226,441, filed on Jul. 28, 2021.

(51) Int. Cl. A61K 31/403; A61K 9/00; A61K 9/08; A61K 47/10; A61P 23/02

(52) U.S. Cl. CPC A61K 31/403; A61K 9/0019; A61K 9/08; A61K 47/10; A61P 23/02

(57) ABSTRACT

The invention provides carbazole derivatives for the treatment of lipoma pain in tissues and organs, in particular treatment of lipoma pain in Dercum's Disease (DD) patients.

### CROSS REFERENCE

This application claims the benefit of U.S. Provisional Application No. 63/226,441, filed on Jul. 28, 2021, which is incorporated herein by reference in its entirety.

### BACKGROUND

Dercum's disease is a rare disorder characterized by multiple, painful growths of fatty tissue (lipomas). The lipomas mainly occur on the trunk, the upper arms and upper legs and are found just below the skin (subcutaneously) but also can be found deeper in the body coupled to muscle, tendons, ligaments or bone by connective tissue Pain associated with Dercum's disease can often be debilitating and resistant to typical analgesic treatments. Pain may be caused by lipomas pressing on nearby nerves or inflamed connective tissue, also called fascia, which is commonly associated with the lipomas. In addition, there are a large number of associated symptoms such as easy bruising, sleep disturbances, impaired memory, depression, difficulty concentrating, anxiety, rapid heartbeat, shortness of breath, diabetes, bloating, constipation, fatigue, joint pain, weight gain, lethargy, and/or confusion.

Dercum's disease mainly occurs in adults and more women are affected than men. There is no approved treatment.

### SUMMARY

The present disclosure provides carbazole derivatives useful for treatment of pain associated with a lipoma. In some embodiments, carbazole derivatives are useful in the treatment of lipoma pain in tissues and organs, in particular treatment of lipoma pain in Dercum's Disease (DD) patients.

In one aspect, provided herein is a method of reducing lipoma pain, the method comprising the step of administering to a subject in need thereof a therapeutically-effective amount of a pharmaceutical composition in unit dosage form, the pharmaceutical compositing comprising a compound of formula (I), or a pharmaceutically-acceptable salt thereof.

In one aspect, provided herein is a pharmaceutical composition in unit dosage form, comprising a compound of formula (I), or a pharmaceutically-acceptable salt thereof, for use in the treatment of lipoma pain.

The compound of Formula (I) is represented by the structure:

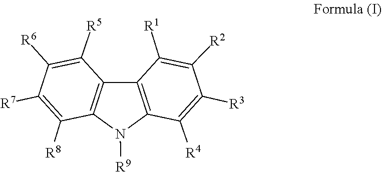

or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(—O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

In some embodiments, treating lipoma pain comprises reducing or alleviating pain associated with a lipoma. In some embodiments, pain is reduced by at least 30% relative to pre-treatment. In some embodiments, pain is reduced by at least 50% relative to pre-treatment. In some embodiments, the lipoma is not an angiolipoma.

In some embodiments, the subject has Dercum's Disease.

In some embodiments, pain is alleviated for at least about 1 month after a single dose of Compound (I). In some embodiments, pain is alleviated for at least about 2 months, at least about 3 months, at least about 4 months, at least about 5 months, at least about 6 months after a single dose of Compound (I), or longer.

In some embodiments, R⁹ is C₁-C₉ alkyl substituted with at least one quaternary ammonium group. In some embodiments, at least one ammonium group is a group of Formula (V):

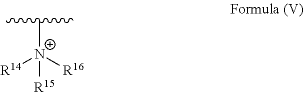

wherein each of R¹⁴, R¹⁵, and R¹⁶ is independently C₁₋₉alkyl, C₂₋₉alkenyl, or C₂₋₉alkynyl.

In some embodiments, the at least one ammonium group is a group of Formula (V′):

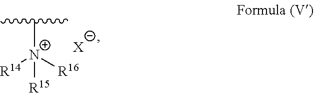

wherein X is a negatively charged ion. In some embodiments, X is a halogen, e.g., Cl.

In some embodiments, each of R¹⁴, R¹⁵, and R¹⁶ is independently methyl. In some embodiments, at least one of R¹, R², R³, and R⁴ is halogen. In some embodiments, at least one of R⁵, R⁶, R⁷, and R⁸ is halogen.

In some embodiments, at least one of R¹, R², R³, and R⁴ is halogen and at least one of R⁵, R⁶, R⁷, and R⁸ is halogen. In some embodiments, the halogen is bromo.

In some embodiments, at least one of R¹, R², R³, and R⁴ is OH. In some embodiments, at least one of R⁵, R⁶, R⁷, and R⁸ is OH. In some embodiments, at least one of R¹, R², R³, and R⁴ is nitro and at least one of R⁵, R⁶, R⁷, and R⁸ is nitro.

In some embodiments, the compound of Formula (I) is: 3-(3,6-dibromo-9H-carbazol-9-yl)-N,N,N-trimethylpropan-1-aminium; 5-(9H-carbazol-9-yl)-N,N,N-trimethylpentan-1-aminium; 5-(2-hydroxy-9H-carbazol-9-yl)-N,N,N-trimethylpentan-1-aminium; or5-(3,6-dibromo-9H-carbazol-9-yl)-N,N,N-trimethylpentan-1-aminium.

In some embodiments, the compound of Formula (I) is represented by the structure of formula (1)

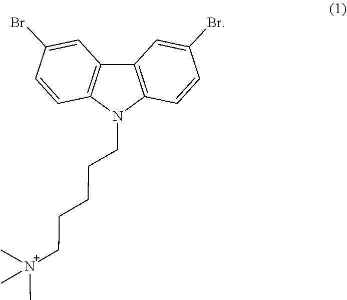 [SMILES: C[N+](C)(C)CCCCCn1c2ccc(Br)cc2c2cc(Br)ccc21]

In some embodiments, the compound of Formula (I) is 5-(3,6-dibromo-9H-carbazol-9-yl)-N,N,N-trimethylpentan-1-aminium chloride.

In some embodiments, the pharmaceutical composition further comprises at least one pharmaceutically-acceptable excipient. In some embodiments, the excipient is a surfactant. In some embodiments, the surfactant is Tween-80. In some embodiments, the excipient is a solubilizing agent. In some embodiments, the solubilizing agent is benzyl alcohol. In some embodiments, the excipient is a solvent. In some embodiments, the solvent is propylene glycol.

In some embodiments, the solvent is water. In some embodiments, the pharmaceutical composition comprises less than about 50% water by weight. In some embodiments, the pharmaceutical composition comprises less than about 30% water by weight. In some embodiments, the pharmaceutical composition comprises less than about 10% water by weight. In some embodiments, the pharmaceutical composition comprises from about 10% to about 30% water by weight.

In some embodiments, the pharmaceutical composition comprises at least about 0.1% by weight of the compound of Formula (I). In some embodiments, the pharmaceutical composition comprises from about 0.1% to about 10% by weight of the compound of Formula (I). In some embodiments, the pharmaceutical composition comprises from about 1% to about 5% by weight of the compound of Formula (I).

In some embodiments, the pharmaceutical composition comprises from 1 to 100 mg compound of formula (I) per mL. In some embodiments, the pharmaceutical composition comprises 50 mg of the compound of formula (I) per mL.

In some embodiments, the pharmaceutical composition is formulated in a liquid dosage form.

In some embodiments, the pharmaceutical composition is administered in a single injection. In some embodiments, the pharmaceutical composition is administered in multiple injections.

In some embodiments, the pharmaceutical composition is administered parenterally. In some embodiments, the pharmaceutical composition is administered subcutaneously.

In some embodiments, the pharmaceutical composition is subcutaneously injected directly into a lipoma. In some embodiments, the pharmaceutical composition is subcutaneously injected directly into a lipoma at a dosage of from about 1 mg to about 10 mg per cm of lipoma. In some embodiments, the pharmaceutical composition is subcutaneously injected directly into a lipoma at a dosage of from about 5 mg to about 10 mg per cm of lipoma.

In some embodiments, the pharmaceutical composition further comprises at least one additional active agent.

### Incorporation by Reference

All publications, patents, and patent applications mentioned in this specification are herein incorporated by reference in their entireties to the same extent as if each individual publication, patent, or patent application was specifically and individually indicated to be incorporated by reference.

### DESCRIPTION OF THE DRAWINGS

FIG. 1: percent reduction of pain per lipoma 84 days after treatment of Dercum's Disease patients with Compound 1 or placebo. A statistically significant average pain reduction of 59.06% was seen in the treatment group vs. 37.50% in placebo (p=0.0004, t-test).

FIG. 2: percent reduction of pain per patient 84 days after treatment of Dercum's Disease patients with Compound 1 or placebo. An average pain reduction of 56.37% was seen in the treatment group vs. 36.82% in placebo.

### DETAILED DESCRIPTION OF THE INVENTION

### Definitions

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

The term “C_{x-y},” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to nine carbon atoms (C₁₋₉ alkyl), one to eight carbon atoms (C₁₋₈ alkyl), one to six carbon atoms (C₁₋₆ alkyl), one to five carbon atoms (C₁₋₅ alkyl), and the like. Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group as defined herein that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂₋₁₂ alkenyl), such as two to nine carbon atoms (C₂₋₉ alkenyl), two to eight carbon atoms (C₂₋₈ alkenyl), two to six carbon atoms (C₂₋₆ alkenyl), two to five carbon atoms (C₂₋₅ alkenyl), and the like. Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂₋₁₂ alkynyl), such as two to nine carbon atoms (C₂₋₉ alkynyl), two to eight carbon atoms (C₂₋₈ alkynyl), two to six carbon atoms (C₂₋₆ alkynyl), two to five carbon atoms (C₂₋₅ alkynyl), and the like. Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon. Exemplary skeletal chain atoms selected from an atom other than carbon include, e.g., O, N, P, Si, S, or combinations thereof, wherein the nitrogen (N), phosphorus (P), or sulfur(S) atoms may optionally be oxidized, and the nitrogen heteroatom may optionally be quaternized (i.e., forming a quaternary ammonium ion). If given, a numerical range refers to the chain length in total. For example, a 3- to 8-membered heteroalkyl has a chain length of 3 to 8 atoms. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein.

“Aryl” refers to an aromatic ring wherein each of the atoms forming the ring is a carbon atom. As used herein, the aryl ring may be selected from monocyclic, bicyclic, tricyclic or multicyclic ring systems wherein at least one of the rings in the ring system is aromatic, i.e., it contains a cyclic, delocalized (4n+2) π-electron system in accordance with the Hückel theory. Aryl groups can be optionally substituted. Examples of aryl groups include, but are not limited to, phenyl naphthyl, fluorenyl, indanyl, indenyl, and tetralinyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted by one or more substituents such as those substituents described herein.

“Heteroaryl” refers to a 3- to 12-membered aromatic ring that comprises at least one heteroatom wherein each heteroatom may be independently selected from N, O, and S. As used herein, the heteroaryl ring may be selected from monocyclic or bicyclic and fused or bridged ring systems wherein at least one of the rings in the ring system is aromatic, i.e., it contains a cyclic, delocalized (4n+2) π-electron system in accordance with the Hückel theory. The heteroatom(s) in the heteroaryl may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, a heteroaryl is optionally substituted by one or more substituents such as those substituents described herein.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms (e.g., C₃₋₁₀ cycloalkyl), such as three to nine carbon atoms (C₃₋₉ cycloalkyl), three to eight carbon atoms (C₃₋₈ cycloalkyl), three to six carbon atoms (C₃₋₆ cycloalkyl), three to five carbon atoms (C₃₋₅ cycloalkyl), and the like. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted by one or more substituents such as those substituents described herein.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical may be partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted by one or more substituents such as those substituents described herein.

The term “substituted” refers to moieties having one or more substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In some embodiments, examples of substituents include, but are not limited to, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O — is equivalent to —OCH₂—.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Compounds of the present disclosure also include crystalline and amorphous forms of those compounds, pharmaceutically-acceptable salts, zwitterions, prodrugs and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)— or (S)—. The present chemical entities, pharmaceutical compositions and methods are meant to include all such possible stereoisomers, including racemic mixtures, optically pure forms, mixtures of diastereomers and intermediate mixtures. Optically active (R)— and (S)— isomers can be prepared using chiral synthons or chiral reagents or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z-or E-form (or cis-or trans-form). Furthermore, some chemical entities may exist in various tautomeric forms. Unless otherwise specified, chemical entities described herein are intended to include all Z-, E- and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E-forms (or cis- and trans-forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The term “effective amount” or “therapeutically effective amount” refers to that amount of a compound described herein that is sufficient to affect the intended application, including but not limited to disease treatment, including alleviation of symptoms, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

### Compounds

Provided herein, in one aspect, is a compound of Formula (I):

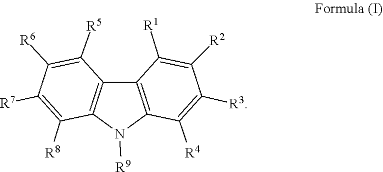

or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(—O)NR¹¹R¹², —NR¹³S(═O)₂R¹⁰, —NR¹³S(—O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

In some embodiments, each of R¹, R², R³, R⁴, R⁵, R⁶, R⁷, and R⁸ is independently selected from the group consisting of H, halogen, —CN, —NO₂, —OR¹⁰, —S(═O)₂R¹⁰, —NR¹¹R¹², —C(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —S(═O)₂R¹⁰, —NR¹¹R¹², —C(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, and —NR¹³S(═O)₂NR¹¹R¹².

In some embodiments, each of R¹, R², R³, R⁴, R⁵, R⁶, R⁷, and R⁸ is independently selected from the group consisting of H, halogen, —CN, —NO₂, —OR¹⁰, —NR¹¹R¹², —C(═O)R¹⁰, —C(═O)OR¹⁰, and C₁₋₅alkyl; wherein each alkyl is independently optionally substituted with one or more substituents selected from halogen, —CN, —NO₂, —OR¹⁰, —S(═O)₂R¹⁰, —NR¹¹R¹², —C(—O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, and —NR¹³S(═O)₂NR¹¹R¹².

In some embodiments, each of R¹, R², R³, R⁴, R⁵, R⁶, R⁷, and R⁸ is independently selected from the group consisting of H, halogen, —CN, —NO₂, —OR¹⁰, and —NR¹¹R¹².

In some embodiments, each of R¹, R², R³, R⁴, R⁵, R⁶, R⁷, and R⁸ is hydrogen.

In some embodiments, at least one of R¹, R², R³, and R⁴ is halogen. In some embodiments, R¹ is halogen. In some embodiments, R² is halogen. In some embodiments, R³ is halogen. In some embodiments, R⁴ is halogen. In some embodiments, at least one of R⁵, R⁶, R⁷, and R⁸ is halogen. In some embodiments, R⁵ is halogen. In some embodiments, R⁶ is halogen. In some embodiments, R⁷ is halogen. In some embodiments, R⁸ is halogen. In some embodiments, at least one of R¹, R², R³, and R⁴ is halogen and at least one of R⁵, R⁶, R⁷, and R⁸ is halogen. In some embodiments, at least one of R¹, R², R³, and R⁴ is halogen, at least one of R⁵, R⁶, R⁷, and R⁸ is halogen, and the rest of R¹, R², R³, R⁴, R⁵, R⁶, R⁷, and R⁸ are hydrogen. In some embodiments, R¹ is halogen and R⁵ is halogen. In some embodiments, R¹ is halogen and R⁶ is halogen. In some embodiments, R¹ is halogen and R⁷ is halogen. In some embodiments, R¹ is halogen and R⁸ is halogen. In some embodiments, R² is halogen and R⁵ is halogen. In some embodiments, R² is halogen and R⁶ is halogen. In some embodiments, R² is halogen and R⁷ is halogen. In some embodiments, R² is halogen and R⁸ is halogen. In some embodiments, R³ is halogen and R⁵ is halogen. In some embodiments, R³ is halogen and R⁶ is halogen. In some embodiments, R³ is halogen and R⁷ is halogen. In some embodiments, R³ is halogen and R⁸ is halogen. In some embodiments, R⁴ is halogen and R⁵ is halogen. In some embodiments, R⁴ is halogen and R⁶ is halogen. In some embodiments, R⁴ is halogen and R⁷ is halogen. In some embodiments, R⁴ is halogen and R⁸ is halogen.

In some embodiments, the halogen is bromo. In some embodiments, the halogen is chloro. In some embodiments, the halogen is fluoro. In some embodiments, the halogen is iodo.

In some embodiments, at least one of R¹, R², R³, and R⁴ is OH. In some embodiments, R¹ is OH. In some embodiments, R² is OH. In some embodiments, R³ is OH. In some embodiments, R⁴ is OH. In some embodiments, at least one of R⁵, R⁶, R⁷, and R⁸ is OH. In some embodiments, R⁵ is OH. In some embodiments, R⁶ is OH. In some embodiments, R⁷ is OH. In some embodiments, R⁸ is OH. In some embodiments, at least one of R¹, R², R³, R⁴, R⁵, R⁶, R⁷, and R⁸ is OH, and the rest of R¹, R², R³, R⁴, R⁵, R⁶, R⁷, and R⁸ are hydrogen.

In some embodiments, at least one of R¹, R², R³, and R⁴ is nitro and at least one of R⁵, R⁶, R⁷, and R⁸ is nitro. In some embodiments, at least one of R¹, R², R³, R⁴, R⁵, R⁶, R⁷, and R⁸ is nitro, and the rest of R¹, R², R³, R⁴, R⁵, R⁶, R⁷, and R⁸ are hydrogen. In some embodiments, R¹ is nitro and R⁵ is nitro. In some embodiments, R¹ is nitro and R⁶ is nitro. In some embodiments, R¹ is nitro and R⁷ is nitro. In some embodiments, R¹ is nitro and R⁸ is nitro. In some embodiments, R² is halogen and R⁵ is nitro. In some embodiments, R² is nitro and R⁶ is nitro. In some embodiments, R² is halogen and R⁷ is nitro. In some embodiments, R² is nitro and R⁸ is nitro. In some embodiments, R³ is halogen and R⁵ is nitro. In some embodiments, R³ is nitro and R⁶ is nitro. In some embodiments, R³ is halogen and R⁷ is nitro. In some embodiments, R³ is nitro and R⁸ is nitro. In some embodiments, R⁴ is halogen and R⁵ is nitro. In some embodiments, R⁴ is nitro and R⁶ is nitro. In some embodiments, R⁴ is halogen and R⁷ is nitro. In some embodiments, R⁴ is nitro and R⁸ is nitro.

In some embodiments, R⁹ is selected from the group consisting of C₁₋₉alkyl, C₂₋₉alkenyl, C₂₋₉alkynyl, and 3- to 10-membered heterocycloalkyl. In some embodiments, R⁹ is C₁₋₉alkyl which is optionally substituted. In some embodiments, R⁹ is a C₁₋₉alkyl which is substituted with at least one quaternary ammonium group. In some embodiments, R⁹ is a C₁₋₉alkyl which is substituted with at least one phosphonium group. In some embodiments, R⁹ is C₂₋₉alkenyl substituted with at least one phosphonium group. In some embodiments, R⁹ is C₂₋₉alkenyl substituted with at least one quaternary ammonium group. In some embodiments, R⁹ is C₂₋₉alkynyl substituted with at least one phosphonium group. In some embodiments, R⁹ is C₂₋₉alkynyl substituted with at least one quaternary ammonium group. In some embodiments, R⁹ is a 3- to 10-membered heterocycloalkyl. In some embodiments, R⁹ is piperazinyl. In some embodiments, R⁹ is pyridinyl. In some embodiments, R⁹ is piperidinyl. In some embodiments, R⁹ is morpholinyl. In some embodiments, R⁹ is thiomorpholinyl. In some embodiments, R⁶ is C₁₋₉alkyl substituted with at least one phosphonium group. In some embodiments, R⁹ is C₁₋₉alkyl substituted with at least one quaternary ammonium group. In some embodiments, R⁹ is propyl substituted with at least one quaternary ammonium group. In some embodiments, R⁹ is butyl substituted with at least one quaternary ammonium group. In some embodiments, R⁹ is pentyl substituted with at least one quaternary ammonium group.

In some embodiments, a compound comprises a positively charged moiety (e.g., ammonium, phosphonium). In some embodiments, a compound which comprises a positively charged moiety may be in the form of a salt, further comprising a negatively-charged counter-ion. For example, when a compound comprises a quaternary ammonium or a phosphonium salt, the compound may be in the form of a salt with a negatively-charged counter-ion, such as a halide (e.g., chloride).

In some embodiments, a compound comprises a negatively charged moiety. In some embodiments, a compound which comprises a negatively charged moiety may be in the form of a salt, further comprising a positively-charged counter-ion.

In some embodiments, the at least one quaternary ammonium group is represented by the structure of Formula (V):

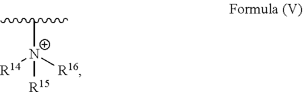

wherein each of R¹⁴, R¹⁵, and R¹⁶ is independently selected from the group consisting of C₁₋₉alkyl, C₂₋₉alkenyl, and C₂₋₉alkynyl. In some embodiments, each of R¹⁴, R¹⁵, and R¹⁶ is independently C₂₋₉alkenyl. In some embodiments, each of R¹⁴, R¹⁵, and R¹⁶ is independently C₂₋₉alkynyl. In some embodiments, each of R¹⁴, R¹⁵, and R¹⁶ is independently C₁₋₉alkyl. In some embodiments, each of R¹⁴, R¹⁵, and R¹⁶ is methyl. In some embodiments, Formula (V) further comprises a counter-ion, as is represented by the structure of formula (V′):

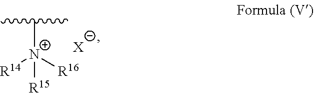

wherein X^{Θ} is a negatively charged counter-ion as defined herein. In some embodiments, X is halogen, e.g., Cl, Br, F, I. In one embodiment, X is a halide, e.g., Cl.

In some embodiments, the at least one phosphonium group is of Formula (VI):

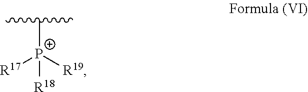

wherein each of R¹⁷, R¹⁸, and R¹⁹ is independently selected from the group consisting of C₁₋₉alkyl, C₂₋₉alkenyl, and C₂₋₉alkynyl. In some embodiments, each of R¹⁷, R¹⁸, and R¹⁹ is independently C₂₋₉alkenyl. In some embodiments, each of R¹⁷, R¹⁸, and R¹⁹ is independently C₂₋₉alkynyl. In some embodiments, each of R¹⁷, R¹⁸, and R¹⁹ is independently C₁₋₉alkyl. In some embodiments, each of R¹⁷, R¹⁸, and R¹⁹ is methyl. In some embodiments, Formula (VI) further comprises a counter-ion, as is represented by the structure of formula (VI′):

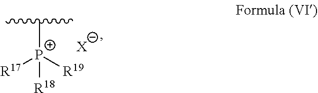

wherein X^{→} is a negatively charged counter-ion as defined herein. In some embodiments, X is halogen, e.g., Cl, Br, F, I. In one embodiment, X is a halide, e.g., Cl.

In some embodiments, R¹⁰ is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, R¹⁰ is independently selected from H, C₁₋₅alkyl, C₁₋₅heteroalkyl, C₁₋₅haloalkyl, and C₃₋₆cycloalkyl. In some embodiments, R¹⁰ is independently selected from H, C₁₋₅alkyl, and C₃₋₆cycloalkyl. In some embodiments, R¹⁰ is H. In some embodiments, R¹⁰ is independently C₁₋₅alkyl. In some embodiments, R¹⁰ is independently C₂₋₅alkenyl. In some embodiments, R¹⁰ is independently C₂₋₅alkynyl. In some embodiments, R¹⁰ is independently C₁₋₅heteroalkyl. In some embodiments, R¹⁰ is independently C₁₋₅haloalkyl. In some embodiments, R¹⁰ is independently C₃₋₆cycloalkyl.

In some embodiments, R¹¹ and R¹² is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, R¹¹ and R¹² together along with the nitrogen atom to which they are attached may form a 3- to 10-membered heterocycloalkyl, which may optionally be substituted. In some embodiments, R¹¹ and R¹² are each independently selected from the group consisting of H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, each of R¹¹ and R¹² is independently selected from H, C₁₋₅alkyl, C₁₋₅heteroalkyl, C₁₋₅haloalkyl, and C₃₋₆cycloalkyl. In some embodiments, each of R¹¹ and R¹² is independently selected from H, C₁₋₅alkyl, and C₃₋₆cycloalkyl. In some embodiments, R¹¹ and R¹² are both H. In some embodiments, each R¹¹ and R¹² is independently C₁₋₅alkyl. In some embodiments, each R¹¹ and R¹² is independently C₂₋₅alkenyl. In some embodiments, each R¹¹ and R¹² is independently C₂₋₅alkynyl. In some embodiments, each R¹¹ and R¹² is independently C₁₋₅heteroalkyl. In some embodiments, each R¹¹ and R¹² is independently C₁₋₅haloalkyl. In some embodiments, each R¹¹ and R¹² is independently C₃₋₆cycloalkyl. In some embodiments, R¹¹ and R¹² are taken together along with the nitrogen atom to which they are attached to form a 3- to 10-membered heterocycloalkyl, which may optionally be substituted.

In some embodiments, R¹³ is independently selected from H, C¹⁻⁵alkyl, C²⁻⁵alkenyl, C₂₋₅alkynyl, C₁₋₅heteroalkyl, C₁₋₅haloalkyl, and C₃₋₆cycloalkyl. In some embodiments, R¹³ is independently selected from H, C₁₋₅alkyl, C₁₋₅heteroalkyl, C₁₋₅haloalkyl, and C₃₋₆cycloalkyl. In some embodiments, R¹³ is independently selected from H, C₁₋₅alkyl, and C₃₋₆cycloalkyl. In some embodiments, R¹³ is H. In some embodiments, R¹³ is independently C₁₋₅alkyl. In some embodiments, R¹³ is independently C₂₋₅alkenyl. In some embodiments, R¹³ is independently C₂₋₅alkynyl. In some embodiments, R¹³ is independently C₁₋₅heteroalkyl. In some embodiments, R¹³ is independently C₁₋₅haloalkyl. In some embodiments, R¹³ is independently C₃₋₆cycloalkyl.

In some embodiments, the compound of Formula (I) is selected from the group consisting of:
3-(3,6-dibromo-9H-carbazol-9-yl)-N,N,N-trimethylpropan-1-aminium,
5-(9H-carbazol-9-yl)-N,N,N-trimethylpentan-1-aminium,
5-(2-hydroxy-9H-carbazol-9-yl)-N,N,N-trimethylpentan-1-aminium, and
5-(3,6-dibromo-9H-carbazol-9-yl)-N,N,N-trimethylpentan-1-aminium.

In some embodiments, the compound of Formula (I) is 3-(3,6-dibromo-9H-carbazol-9-yl)-N,N,N-trimethylpropan-1-aminium. In some embodiments, the compound of Formula (I) is 5-(9H-carbazol-9-yl)-N,N,N-trimethylpentan-1-aminium. In some embodiments, the compound of Formula (I) is 5-(2-hydroxy-9H-carbazol-9-yl)-N,N,N-trimethylpentan-1-aminium. In some embodiments, the compound of Formula (I) is 5-(3,6-dibromo-9H-carbazol-9-yl)-N,N,N-trimethylpentan-1-aminium.

In some embodiments, the compound of formula (I) is in a pharmaceutically-acceptable salt form. The term “salt” or “pharmaceutically-acceptable salt” refers to salts derived from a variety of organic and inorganic counter-ions. When a compound of formula (I) contains one or more positive charges, the counter-ion has the corresponding one or more negative charges, generating a neutral molecule. When a compound of formula (I) contains one or more negative charges, the counter-ion has the corresponding one or more positive charges, generating a neutral molecule.

Pharmaceutically-acceptable acid addition salts can be formed with inorganic acids and organic acids and their corresponding counter-ions. Inorganic acids from which salts can be derived include, for example, hydrochloric acid, hydrobromic acid, sulfuric acid, nitric acid, phosphoric acid, and the like. For example, salts may include a counter anion being a halogen counter-anion such as for example chloride and bromide anions. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically-acceptable base addition salts can be formed with inorganic and organic bases and their corresponding counter-ions. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically-acceptable base addition salt is chosen from ammonium, phosphonium, potassium, sodium, calcium, and magnesium salts.

In some embodiments, Compound (I) is in the form of a salt with an inorganic acid. In some embodiments, Compound (I) is in the form of a salt with a hydrochloric acid (i.e., the counter-ion is chloride (Cl⁻)).

In some embodiments, the compound of Formula (I) is 5-(3,6-dibromo-9H-carbazol-9-yl)-N,N,N-trimethylpentan-1-aminium, represented by the structure of formula 1:

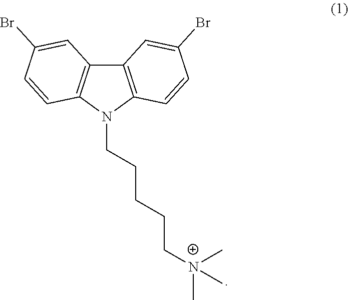 [SMILES: C[N+](C)(C)CCCCCn1c2ccc(Br)cc2c2cc(Br)ccc21]

In some embodiments, the compound of Formula (I) is 5-(3,6-dibromo-9H-carbazol-9-yl)-N,N,N-trimethylpentan-1-aminium chloride, represented by the structure of formula 1A:

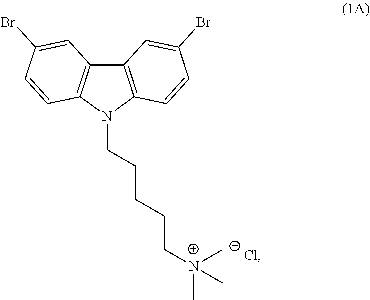

In some embodiments, the compound of Formula (I) is represented by the structure of formula (2):

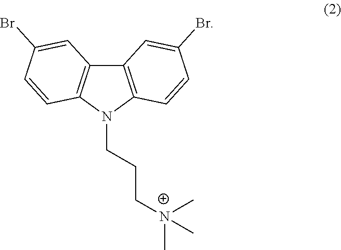 [SMILES: C[N+](C)(C)CCCn1c2ccc(Br)cc2c2cc(Br)ccc21]

In some embodiments, the compound of Formula (I) is represented by the structure of formula (3):

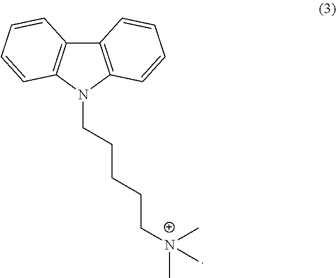 [SMILES: C[N+](C)(C)CCCCCn1c2ccccc2c2ccccc21]

In some embodiments, the compound of Formula (I) is represented by the structure of formula

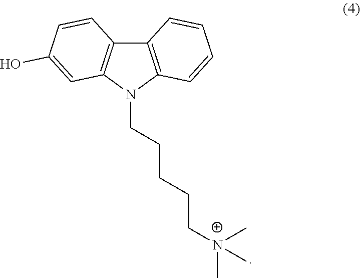 [SMILES: C[N+](C)(C)CCCCCn1c2ccccc2c2ccc(O)cc21]

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Methods of Use

In some embodiments, the compounds and compositions described herein are useful for treating pain. In some embodiments, the compounds and compositions described herein are useful for treating pain that is associated with a lipoma (i.e., a lipoma pain). In some embodiments, the compounds and compositions described herein are useful for treating pain that is associated with a lipoma in a subject having Dercum's Disease. As shown herein, injection of a single dose of Compound 1 (either via a single injection or multiple injections) into lipomas significantly alleviates lipoma-associated pain.

In some embodiments, the subject being treated has Dercum's Disease. In some embodiments, the subject has lipomas that are associated with Dercum's disease. Dercum's Disease is a rare adipose disorder (RAD) and is typically characterized by obesity and chronic pain (>3 months) in the subcutaneous adipose tissue (SAT). The pain associated with DD is often debilitating and resistant to typical analgesic treatments.

The etiology of Dercum's Disease is currently unknown. Several hypotheses have been proposed including nervous system dysfunction, adipose tissue dysfunction, lymphovascular disorder, endocrine dysfunction, mechanical pressure on nerves, and a result of trauma. A few reports suggest that Dercum's Disease can be an inherited autosomal dominant disorder with variable expression, however the majority of cases occur sporadically without any specific genetic mutations.

Dercum's Disease can be classified into 4 types which include: generalized diffuse, generalized nodular, localized nodular, and juxta-articular forms. The diffuse type is characterized by widespread pain from SATs located anywhere from the head to the soles of the feet, without any clear lipomas. Generalized nodular typically occurs with widespread, painful adipose tissue that is more painful in the vicinity of lipomas, whereas in the localized nodular type, the pain is confined to areas within and around lipomas. Finally, the juxta-articular type is characterized by painful folds or nodular fat around joints such as the knees and/or the hips The carbazole derivatives of the present disclosure (e.g., compound I, 1, 2, 3 or 4) are useful in treating any one or more of the types of Dercum's Disease, including generalized diffuse, generalized nodular, localized nodular, and juxta-articular forms.

There are Dercum's subjects who have mixed lipomas, i.e. small and large lipomas. Lipomas may range from less than 1 cm in diameter to more than 10 cm in diameter. In some embodiments, lipomas treatable by the compounds of the disclosure range from 1-2 cm, 2-3 cm, 3-4 cm, 4-5 cm, 5-6 cm, 6-7 cm, 7-8 cm, 8-9 cm, 9-10 cm, 1-3 cm, 2-4 cm, 3-5 cm, 4-6 cm, 5-7 cm, 6-8 cm, 7-9 cm, 8-10 cm, for example 1, 2, 3, 4, 5, 6, 7, 8, 9, 10 cm in diameter, or shorter or longer. As described herein, a dose of the carbazole derivatives of the present disclosure (e.g., compound I, 1, 2, 3 or 4) may be adjusted based on the size of the lipoma being treated, and/or the degree of pain experienced by the subject being treated.

In some embodiments, provided herein is a method of treating lipoma pain, the method comprising the step of administering to a subject in need thereof a therapeutically-effective amount of a pharmaceutical composition in unit dosage form, the pharmaceutical compositing comprising a compound of formula (I):

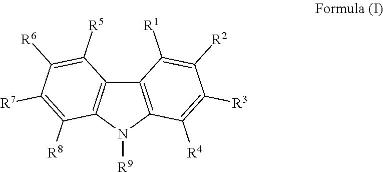

or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(—O)₂R¹⁰, —NR¹¹R¹², —C(═O)NR¹¹R¹², —S(═O)NR¹¹R¹², S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S (═O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(—O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², C(═S)R¹⁰, —N(═O), —SN(═O), —NR¹³N(═O), and —ON (═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

In some embodiments, provided herein is a method of treating lipoma pain, the method comprising the step of administering to a subject in need thereof a therapeutically-effective amount of a pharmaceutical composition in unit dosage form, the pharmaceutical compositing comprising a compound of formula 1, or a pharmaceutically-acceptable salt thereof. In some embodiments, provided herein is a method of treating lipoma pain, the method comprising the step of administering to a subject in need thereof a therapeutically-effective amount of a pharmaceutical composition in unit dosage form, the pharmaceutical compositing comprising a compound of formula 2, or a pharmaceutically-acceptable salt thereof. In some embodiments, provided herein is a method of treating lipoma pain, the method comprising the step of administering to a subject in need thereof a therapeutically-effective amount of a pharmaceutical composition in unit dosage form, the pharmaceutical compositing comprising a compound of formula 3, or a pharmaceutically-acceptable salt thereof. In some embodiments, provided herein is a method of treating lipoma pain, the method comprising the step of administering to a subject in need thereof a therapeutically-effective amount of a pharmaceutical composition in unit dosage form, the pharmaceutical compositing comprising a compound of formula 4, or a pharmaceutically-acceptable salt thereof.

Provided herein is a pharmaceutical composition in unit dosage form, the pharmaceutical compositing comprising a compound of formula (I), for use in the treatment of lipoma pain, e.g., pain associated with Dercum's Disease lipomas. Provided herein is a pharmaceutical composition in unit dosage form, the pharmaceutical compositing comprising a compound of formula 1, for use in the treatment of lipoma pain, e.g., pain associated with Dercum's Disease lipomas. Provided herein is a pharmaceutical composition in unit dosage form, the pharmaceutical compositing comprising a compound of formula 2, for use in the treatment of lipoma pain, e.g., pain associated with Dercum's Disease lipomas. Provided herein is a pharmaceutical composition in unit dosage form, the pharmaceutical compositing comprising a compound of formula 3, for use in the treatment of lipoma pain, e.g., pain associated with Dercum's Disease lipomas. Provided herein is a pharmaceutical composition in unit dosage form, the pharmaceutical compositing comprising a compound of formula 4, for use in the treatment of lipoma pain, e.g., pain associated with Dercum's Disease lipomas.

Compounds of the disclosure reduce or eliminate pain associated with lipomas. In some embodiments, pain is reduced by at least 10% relative to pre-treatment, e.g., by about 15%, 20%, 25%, 30%, 35%, 40%, 45%, 50%, 55%, 60%, 65%, 70%, 75%, 80%, 85%, 90%, 95% or 100% relative to pre-treatment. In some embodiments, pain is reduced by at least 10%, 15%, 20%, 25%, 30%, 35%, 40%, 45%, 50%, 55%, 60%, 65%, 70%, 75%, 80%, 85%, 90%, 95% or 100% relative to pre-treatment after a single dose of the compound of formula (I), wherein the single dose may be given in a single injection or multiple injections, as described herein.

In some embodiments, reduction in pain is measured by a comparative pain scale, which ranks pain from a score from 0 to 10. For example, a score of 0 may indicate no pain, a score of 1-3 may indicate minor pain, a score of 4-6 may indicate moderate pain and a score of 7-10 may indicate severe pain. In some embodiments, reduction in pain is evaluated per subject, as described herein. In some embodiments, reduction in pain is evaluated per subject, relative to pre-treatment. In some embodiments, reduction in pain is evaluated per subject, relative to placebo control. In some embodiments, reduction in pain is measured/analyzed per lipoma, as described herein. In some embodiments, reduction in pain is measured/analyzed per lipoma, relative to pre-treatment. In some embodiments, reduction in pain is measured/analyzed per lipoma, relative to placebo control.

In some embodiments, the lipoma is not an angiolipoma. Angiolipomas are subcutaneous tumors of the extremities and trunk. Subcutaneous angiolipomas have a normal karyotype, setting them apart from most other fatty tumors, including lipomas. For this reason, they have been regarded as a hamartoma of blood vessels and fat, rather than as a true fatty tumor. In contrast to lipomas that contain only fat tissue, angiolipomas have a thin fibrous capsule with incomplete fibrous septa extending into the lesion, dividing it into lobules of different size. They are composed of variable proportions of fatty tissue and blood vessels.

In some embodiments, Compound of the disclosure alleviate lipoma pain for an extended period of time after a single dose of Compound (I). For example, duration of pain may be alleviated for at least about 1 month after a single dose of Compound (I). In some embodiments, pain may be alleviated for at least about 2 months, at least about 3 months, at least about 4 months, at least about 5 months, at least about 6 months after a single dose of Compound (I), or longer. For example, duration of pain may be alleviated for at least about 30 days after a single dose of Compound (I). In some embodiments, pain may be alleviated for at least about 60 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 120 days, 150 days, 180 days, 200 days, 220 days, 240 days, 260 days, 280 days, 300 days, 320 days, 340 days, 360 days or 365 days after a single dose of Compound (I), or even longer. It is understood that a single administration of compound (I) can be provided in a single injection or multiple injections of a dose of compound (I).

### Dosing and Dosing Regimens

In some embodiments, the pharmaceutical composition is formulated for parenteral administration. In some embodiments, the pharmaceutical composition is formulated for subcutaneous injection directly into a lipoma. In some embodiments, injections may be spread randomly on the lipoma surface with a distance between injections of at least 1 cm, e.g., 1.1 cm, 1.2 cm, 1.3 cm, 1.4 cm, 1.5 cm, 1.6 cm, 1.7 cm, 1.8 cm, 1.9 cm, 2.0 cm or even longer. In some embodiments, injections may be given at 90° to the injected skin surface or at any other angle as desired, e.g., 5°, 10°, 15°, 20°, 25°, 30°, 35°, 40° 45°, 50°, 55°, 60°, 65°, 70°, 75°, 80° or 85°. In some embodiments, at least 1, 2, 3, 4, 5,6, 7, 8, 9, 10 or even more lipomas/nodules may be injected per subject.

In some embodiments, the pharmaceutical composition is formulated for subcutaneous injection directly into an lipoma at a dosage of from about 1 to about 10 mg compound (I) (e.g., Compound 1, 2, 3, 4) per cm per lipoma (based on lipoma diameter). In some embodiments, the pharmaceutical composition is formulated for subcutaneous injection directly into an lipoma at a dosage of from about 1 to about 50 mg compound (I) (e.g., compound 1, 2, 3, 4) per cm per lipoma (based on lipoma diameter), e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg compound (I) (e.g., compound 1, 2, 3, 4) per cm lipoma (based on lipoma diameter).

In some embodiments, the pharmaceutical composition is administered parenterally. In some embodiments, the pharmaceutical composition is administered subcutaneously. In some embodiments, the pharmaceutical composition is subcutaneously injected directly into a lipoma. In some embodiments, the pharmaceutical composition is subcutaneously injected directly into a lipoma at a dosage of from about 0.05 to about 0.1 mL per lipoma. In some embodiments, the pharmaceutical composition is formulated for subcutaneous injection directly into a lipoma at a dosage of from about 0.05 to about 0.2 mL per lipoma. In some embodiments, the pharmaceutical composition is formulated for subcutaneous injection directly into a lipoma at a dosage of from about 0.1 to about 0.2 mL per lipoma. In some embodiments, the pharmaceutical composition is formulated for subcutaneous injection directly into a lipoma at a dosage of from about 0.1 to about 0.4 mL per lipoma. In some embodiments, the pharmaceutical composition is formulated for subcutaneous injection directly into a lipoma at a dosage of from about 0.4 to about 1 mL per lipoma. In some embodiments, the pharmaceutical composition is formulated for subcutaneous injection directly into a lipoma at a dosage of from about 1 to about 2 mL per lipoma. In some embodiments, the pharmaceutical composition is formulated for subcutaneous injection directly into a lipoma at a dosage of less than about 0.05 mL per lipoma. In some embodiments, the pharmaceutical composition is formulated for subcutaneous injection directly into a lipoma at a dosage of about 0.05 mL per lipoma. In some embodiments, the pharmaceutical composition is formulated for subcutaneous injection directly into a lipoma at a dosage of about 0.1 mL per lipoma. In some embodiments, the pharmaceutical composition is formulated for subcutaneous injection directly into a lipoma at a dosage of about 0.2 mL per lipoma. In some embodiments, the pharmaceutical composition is formulated for subcutaneous injection directly into a lipoma at a dosage of about 0.3 mL per lipoma. In some embodiments, the pharmaceutical composition is formulated for subcutaneous injection directly into a lipoma at a dosage of about 0.4 mL per lipoma. In some embodiments, the pharmaceutical composition is formulated for subcutaneous injection directly into a lipoma at a dosage of about 0.5 mL per lipoma. In some embodiments, the pharmaceutical composition is formulated for subcutaneous injection directly into a lipoma at a dosage of about 0.6 mL per lipoma. In some embodiments, the pharmaceutical composition is formulated for subcutaneous injection directly into a lipoma at a dosage of about 0.7 mL per lipoma. In some embodiments, the pharmaceutical composition is formulated for subcutaneous injection directly into a lipoma at a dosage of about 0.8 mL per lipoma. In some embodiments, the pharmaceutical composition is formulated for subcutaneous injection directly into a lipoma at a dosage of about 0.9 mL per lipoma. In some embodiments, the pharmaceutical composition is formulated for subcutaneous injection directly into a lipoma at a dosage of about 1.0 mL per lipoma. In some embodiments, the pharmaceutical composition is formulated for subcutaneous injection directly into a lipoma at a dosage of about 1.1 mL per lipoma. In some embodiments, the pharmaceutical composition is formulated for subcutaneous injection directly into a lipoma at a dosage of about 1.2 mL per lipoma. In some embodiments, the pharmaceutical composition is formulated for subcutaneous injection directly into a lipoma at a dosage of about 1.3 mL per lipoma. In some embodiments, the pharmaceutical composition is formulated for subcutaneous injection directly into a lipoma at a dosage of about 1.4 mL per lipoma. In some embodiments, the pharmaceutical composition is formulated for subcutaneous injection directly into a lipoma at a dosage of about 1.5 mL per lipoma. In some embodiments, the pharmaceutical composition is formulated for subcutaneous injection directly into a lipoma at a dosage of about 1.6 mL per lipoma. In some embodiments, the pharmaceutical composition is formulated for subcutaneous injection directly into a lipoma at a dosage of about 1.7 mL per lipoma. In some embodiments, the pharmaceutical composition is formulated for subcutaneous injection directly into a lipoma at a dosage of about 1.8 mL per lipoma. In some embodiments, the pharmaceutical composition is formulated for subcutaneous injection directly into a lipoma at a dosage of about 1.9 mL per lipoma. In some embodiments, the pharmaceutical composition is formulated for subcutaneous injection directly into a lipoma at a dosage of about 2.0 mL per lipoma. In some embodiments, the pharmaceutical composition is formulated for subcutaneous injection directly into a lipoma at a dosage of greater than about 2.0 mL per lipoma.

In some embodiments, the pharmaceutical composition is subcutaneously injected directly into a lipoma at a dosage of from about 0.05 to about 0.1 mL per cm lipoma. In some embodiments, the pharmaceutical composition is formulated for subcutaneous injection directly into a lipoma at a dosage of from about 0.05 to about 0.2 mL per cm lipoma. In some embodiments, the pharmaceutical composition is formulated for subcutaneous injection directly into a lipoma at a dosage of from about 0.1 to about 0.2 mL per cm lipoma. In some embodiments, the pharmaceutical composition is formulated for subcutaneous injection directly into a lipoma at a dosage of from about 0.1 to about 0.4 mL per cm lipoma. In some embodiments, the pharmaceutical composition is formulated for subcutaneous injection directly into a lipoma at a dosage of from about 0.4 to about 1 mL per cm lipoma. In some embodiments, the pharmaceutical composition is formulated for subcutaneous injection directly into a lipoma at a dosage of from about 1 to about 2mL per cm lipoma. In some embodiments, the pharmaceutical composition is formulated for subcutaneous injection directly into a lipoma at a dosage of less than about 0.05 mL per cm lipoma. In some embodiments, the pharmaceutical composition is formulated for subcutaneous injection directly into a lipoma at a dosage of about 0.05 mL per cm lipoma. In some embodiments, the pharmaceutical composition is formulated for subcutaneous injection directly into a lipoma at a dosage of about 0.1 mL per cm lipoma. In some embodiments, the pharmaceutical composition is formulated for subcutaneous injection directly into a lipoma at a dosage of about 0.2 mL per cm lipoma. In some embodiments, the pharmaceutical composition is formulated for subcutaneous injection directly into a lipoma at a dosage of about 0.3 mL per cm lipoma. In some embodiments, the pharmaceutical composition is formulated for subcutaneous injection directly into a lipoma at a dosage of about 0.4 mL per cm lipoma. In some embodiments, the pharmaceutical composition is formulated for subcutaneous injection directly into a lipoma at a dosage of about 0.5 mL per cm lipoma. In some embodiments, the pharmaceutical composition is formulated for subcutaneous injection directly into a lipoma at a dosage of about 0.6 mL per cm lipoma. In some embodiments, the pharmaceutical composition is formulated for subcutaneous injection directly into a lipoma at a dosage of about 0.7 mL per cm lipoma. In some embodiments, the pharmaceutical composition is formulated for subcutaneous injection directly into a lipoma at a dosage of about 0.8 mL per cm lipoma. In some embodiments, the pharmaceutical composition is formulated for subcutaneous injection directly into a lipoma at a dosage of about 0.9 mL per cm lipoma. In some embodiments, the pharmaceutical composition is formulated for subcutaneous injection directly into a lipoma at a dosage of about 1.0 mL per cm lipoma. In some embodiments, the pharmaceutical composition is formulated for subcutaneous injection directly into a lipoma at a dosage of about 1.1 mL per cm lipoma. In some embodiments, the pharmaceutical composition is formulated for subcutaneous injection directly into a lipoma at a dosage of about 1.2 mL per cm lipoma. In some embodiments, the pharmaceutical composition is formulated for subcutaneous injection directly into a lipoma at a dosage of about 1.3 mL per cm lipoma. In some embodiments, the pharmaceutical composition is formulated for subcutaneous injection directly into a lipoma at a dosage of about 1.4 mL per cm lipoma. In some embodiments, the pharmaceutical composition is formulated for subcutaneous injection directly into a lipoma at a dosage of about 1.5 mL per cm lipoma. In some embodiments, the pharmaceutical composition is formulated for subcutaneous injection directly into a lipoma at a dosage of about 1.6 mL per cm lipoma. In some embodiments, the pharmaceutical composition is formulated for subcutaneous injection directly into a lipoma at a dosage of about 1.7 mL per cm lipoma. In some embodiments, the pharmaceutical composition is formulated for subcutaneous injection directly into a lipoma at a dosage of about 1.8 mL per cm lipoma. In some embodiments, the pharmaceutical composition is formulated for subcutaneous injection directly into a lipoma at a dosage of about 1.9 mL per cm lipoma. In some embodiments, the pharmaceutical composition is formulated for subcutaneous injection directly into a lipoma at a dosage of about 2.0 mL per cm lipoma. In some embodiments, the pharmaceutical composition is formulated for subcutaneous injection directly into a lipoma at a dosage of greater than about 2.0 mL per cm lipoma.

The total amount of each compound administered will be dependent on the mammal being treated, the severity of the disorder or condition, the rate of administration, the disposition of the compound and the discretion of the prescribing physician. However, an effective dosage may be in the range of about 0.001 to about 100 mg per kg body weight per day, in single or divided doses. In some embodiments, a compound is administered in an amount ranging from about 0.01 mg/kg to about 100 mg/kg, 0.1 mg/kg to about 100 mg/kg, about 10 mg/kg to about 80 mg/kg, about 20 mg/kg to about 50 mg/kg, and the like. In some instances, dosage levels below the lower limit of the aforesaid range may be more than adequate, while in other cases still larger doses may be employed without causing any harmful side effect, e.g., by dividing such larger doses into several small doses for administration throughout the day. In some embodiments, an effective dosage may be provided in pulsed dosing (i.e., administration of the compound in consecutive days, followed by consecutive days of rest from administration).

In some embodiments, the compound of Formula (I) may be administered in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg per subject.

In some embodiments, the pharmaceutical composition is administered in a single dose. In some embodiments, the pharmaceutical composition is administered via a single injection. In some embodiments, the pharmaceutical composition is administered in a single dose via a single injection. In some embodiments, the pharmaceutical composition is administered in a single dose via multiple injections. In some embodiments, the pharmaceutical composition is administered in multiple doses. the pharmaceutical composition is administered in multiple doses via multiple injections.

In some embodiments, the pharmaceutical composition is administered via multiple injections, e.g., 2, 3, 4, 5, 6, 7, 8, 9, 10, 11, 12, 13, 14, or 15 injections per lipoma. In some embodiments, the pharmaceutical composition is administered via multiple injections, e.g., 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 injections per subject. In some embodiments, the pharmaceutical composition is administered in a single dose via multiple injections, e.g., 2, 3, 4, 5, 6, 7, 8, 9, 10, 11, 12, 13, 14, or 15 injections per lipoma. In some embodiments, the pharmaceutical composition is administered in a single dose via multiple injections, e.g., 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 injections per subject.

### Combination Therapy

In some embodiments, carbazole derivatives of the disclosure can be used in combination with one or more additional therapies. In some embodiments, carbazole derivatives of the disclosure can be used in conjunction with medications used as standard of care to treat pain, e.g., lipoma pain, e.g., lipoma pain associated with Dercum's Disease.

No specific treatment exists for Dercum's Disease. Treatment is mainly symptomatic and supportive and is primarily focused on easing the characteristic painful episodes. Various analgesics have been used (i.e., lidocaine). Surgical excision of lipomas may temporarily relieve symptoms although recurrences often develop. Liposuction has been used as a supportive treatment for some individuals with Dercum's Disease, and may provide an initial reduction in pain and improvement in quality of life (QOL). Psychotherapy and consultation with pain management specialists may be helpful for enabling affected individuals to cope with long-term intense pain.

In some embodiments, the compounds or pharmaceutical compositions of the disclosure may be used in conjunction with any one or more of the aforementioned therapies. Thus, in some embodiments, the pharmaceutical compositions of the disclosure further comprise at least one additional active agent. In some embodiments, the additional active agent is a cytotoxic agent. In some embodiments, the additional active agent is an analgesic. An “analgesic” may be any member of the group of drugs used to achieve reduction or relief of pain in mammals. Non-limiting examples include acetaminophen/paracetamol, nonsteroidal anti-inflammatory drugs (NSAIDs), COX-2 inhibitors, opioids, etc.

Thus, in some embodiments, the pharmaceutical composition comprising the compound of Formula (I) can be administered as part of a therapeutic regimen that comprises administering one or more second agents (e.g. 1, 2, 3, 4, 5, or more second agents useful in treating pain, e.g., pain associated with Dercum's lipomas), either simultaneously or sequentially with the pharmaceutical composition comprising the compound of Formula (I). When administered sequentially, the pharmaceutical composition comprising the compound of Formula (I) may be administered before or after the one or more second agents. When administered simultaneously, the pharmaceutical composition comprising the compound of Formula (I) and the one or more second agents may be administered by the same route (e.g. injections to the same location), by a different route (e.g. a tablet taken orally while receiving an intravenous infusion), or as part of the same combination (e.g. a solution comprising the pharmaceutical composition comprising the compound of Formula (I) and one or more second agents).

A combination treatment according to the disclosure may be effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. The exact dosage will depend upon the agent selected, the route of administration, the form in which the compound is administered, the subject to be treated, the body weight of the subject to be treated, and the preference and experience of the attending physician.

### Pharmaceutical Compositions

A composition of the present disclosure may be formulated in any suitable pharmaceutical formulation. A pharmaceutical composition of the present disclosure typically contains an active ingredient (e.g., a compound of Formula (I), or a pharmaceutically-acceptable salt and/or coordination complex thereof), and one or more pharmaceutically-acceptable excipients or carriers, including but not limited to: inert solid diluents and fillers, diluents, sterile aqueous solution and various organic solvents, permeation enhancers, solubilizers, and adjuvants. A composition of the present disclosure may be formulated in any suitable pharmaceutical formulation.

Pharmaceutical compositions may be provided in any suitable form, which may depend on the route of administration. In some embodiments, the pharmaceutical composition disclosed herein can be formulated in dosage form for administration to a subject. In some embodiments, the pharmaceutical composition is formulated for parenteral, topical, transdermal, buccal, sublingual, subcutaneous, intramuscular, intravenous, intratumoral, and/or intraperitoneal administration. In some embodiments, the pharmaceutical composition can be formulated as a unit dosage.

In some embodiments, the composition is provided in one or more unit doses. For example, the composition can be administered in 1, 2, 3, 4, 5, 6, 7, 14, 30, 60, or more doses. Such amount can be administered each day, for example in individual doses administered once, twice, or three or more times a day. However, dosages stated herein on a per day basis should not be construed to require administration of the daily dose each and every day. For example, if one of the agents is provided in a suitably slow-release form, two or more daily dosage amounts can be administered at a lower frequency, e.g., as a depot injection administered every second day to once a month or even longer. Most typically and conveniently for the subject, a pharmaceutical composition comprising a compound of Formula (I) can be administered once a day, for example in the morning, in the evening or during the day.

The unit doses can be administered simultaneously or sequentially. The composition can be administered for an extended treatment period. Illustratively, the treatment period can be at least about one month, for example at least about 3 months, at least about 6 months or at least about 1 year. In some cases, administration can continue for substantially the remainder of the life of the subject.

Pharmaceutical compositions described herein may contain one or more pharmaceutically-acceptable excipients. The phrase “pharmaceutically-acceptable excipient” or “pharmaceutically-acceptable carrier” as used herein means a pharmaceutically-acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically-acceptable carriers include: (1) surfactants such as hydrophilic surfactants, lipophilic surfactants, non-ionic surfactants, cationic surfactants, anionic surfactants; (2) solubilizers such as alcohols; (3) solvents such as water, alcohol and glycols; (4) sugars, such as lactose, glucose and sucrose; (5) starches, such as corn starch and potato starch; (6) cellulose, and its derivatives, such as sodium carboxymethyl cellulose, ethyl cellulose and cellulose acetate; (7) powdered tragacanth; (8) malt; (9) gelatin; (10) talc; (11) excipients, such as cocoa butter and suppository waxes; (12) oils, such as peanut oil, cottonseed oil, safflower oil, sesame oil, olive oil, corn oil and soybean oil; (13) glycols, such as propylene glycol; (14) polyols, such as glycerin, sorbitol, mannitol and polyethylene glycol; (15) esters, such as ethyl oleate and ethyl laurate; (16) agar; (17) buffering agents, such as magnesium hydroxide and aluminum hydroxide; (18) alginic acid; (19) pyrogen-free water; (20) isotonic saline; (21) Ringer's solution; (22) ethyl alcohol; (23) phosphate buffer solutions; and (24) other non-toxic compatible substances employed in pharmaceutical formulations. The composition can further include one or more pharmaceutically-acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In some embodiments, the pharmaceutical composition comprises one or more surfactants. Surfactants which can be used to form pharmaceutical composition and dosage forms of the disclosure include, but are not limited to, hydrophilic surfactants, lipophilic surfactants, non-ionic surfactants, cationic surfactants, anionic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono-and di-acetylated tartaric acid esters of mono-and di-glycerides; succinylated mono-and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono-and di-acetylated tartaric acid esters of mono-and di-glycerides; succinylated mono-and di-glycerides; citric acid esters of mono-and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, Tween 80, sucrose monostearate, sucrose monolaurate, sucrose monopalmitate, PEG 10-100 nonyl phenol series, PEG 15-100 octyl phenol series, and poloxamers. In some embodiments, the surfactant is Tween. In some embodiments, the surfactant is Tween 80.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono-and di-glycerides; hydrophobic transesterification products of a polyol with at least one member of the group of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group of vegetable oils, hydrogenated vegetable oils, and triglycerides.

In one embodiment, the composition may include a solvent/solubilizer to ensure good solubilization and/or dissolution of the compound of the present disclosure and to minimize precipitation of the compound of the present disclosure. This can be especially important for injection. A solubilizer may also be added to increase the solubility of the hydrophilic drug and/or other components, such as surfactants, or to maintain the composition as a stable or homogeneous solution or dispersion. Examples of suitable solvents/solubilizers include, but are not limited to: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water. In some embodiments, the solubilizer is benzyl alcohol.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. If present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

In addition, an acid or a base may be incorporated into the composition to facilitate processing, to enhance stability, or for other reasons. Examples of pharmaceutically-acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically-acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically-acceptable cation, such as ammonium, alkali metals, alkaline earth metals, and the like. Example may include, but not limited to, sodium, potassium, lithium, magnesium, calcium and ammonium.

Suitable acids for use in the compositions of the disclosure are pharmaceutically-acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Provided herein, in one aspect, is a pharmaceutical composition comprising a compound of Formula (I):

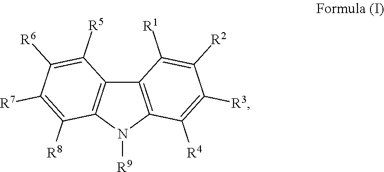

or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S) R¹⁰, —N(═O), —SN(═O), —NR¹³N(═O), ON(—O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(—O)R¹⁰, —S(—O)₂R¹⁰, —NR¹¹R¹², —C(═O)NR¹¹R¹², —S(═O)NR¹¹R¹², —S(═O)₂NR¹¹R¹², —C (═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

In some embodiments, the pharmaceutical composition comprises less than about 50% water by weight. In some embodiments, the pharmaceutical composition comprises less than about 30% water by weight. In some embodiments, the pharmaceutical composition comprises less than about 10% water by weight. In some embodiments, the pharmaceutical composition comprises from about 0% to about 30% water by weight. In some embodiments, the pharmaceutical composition comprises from about 10% to about 30% water by weight. In some embodiments, the pharmaceutical composition comprises from about 15% to about 30% water by weight. In some embodiments, the pharmaceutical composition comprises from about 15% to about 25% water by weight. In some embodiments, the pharmaceutical composition comprises from about 20% to about 30% water by weight. In some embodiments, the pharmaceutical composition comprises from about 23% to about 27% water by weight. In some embodiments, the pharmaceutical composition comprises from about 24% to about 26% water by weight. In some embodiments, the pharmaceutical composition comprises about 0% water by weight. In some embodiments, the pharmaceutical composition comprises about 1% water by weight. In some embodiments, the pharmaceutical composition comprises about 2% water by weight. In some embodiments, the pharmaceutical composition comprises about 3% water by weight. In some embodiments, the pharmaceutical composition comprises about 4% water by weight. In some embodiments, the pharmaceutical composition comprises about 5% water by weight. In some embodiments, the pharmaceutical composition comprises about 6% water by weight. In some embodiments, the pharmaceutical composition comprises about 7% water by weight. In some embodiments, the pharmaceutical composition comprises about 8% water by weight. In some embodiments, the pharmaceutical composition comprises about 9% water by weight. In some embodiments, the pharmaceutical composition comprises about 10% water by weight. In some embodiments, the pharmaceutical composition comprises about 11% water by weight. In some embodiments, the pharmaceutical composition comprises about 12% water by weight. In some embodiments, the pharmaceutical composition comprises about 13% water by weight. In some embodiments, the pharmaceutical composition comprises about 14% water by weight. In some embodiments, the pharmaceutical composition comprises about 15% water by weight. In some embodiments, the pharmaceutical composition comprises about 16% water by weight. In some embodiments, the pharmaceutical composition comprises about 17% water by weight. In some embodiments, the pharmaceutical composition comprises about 18% water by weight. In some embodiments, the pharmaceutical composition comprises about 19% water by weight. In some embodiments, the pharmaceutical composition comprises about 20% water by weight. In some embodiments, the pharmaceutical composition comprises about 21% water by weight. In some embodiments, the pharmaceutical composition comprises about 22% water by weight. In some embodiments, the pharmaceutical composition comprises about 23% water by weight. In some embodiments, the pharmaceutical composition comprises about 24% water by weight. In some embodiments, the pharmaceutical composition comprises about 25% water by weight. In some embodiments, the pharmaceutical composition comprises about 26% water by weight. In some embodiments, the pharmaceutical composition comprises about 27% water by weight. In some embodiments, the pharmaceutical composition comprises about 28% water by weight. In some embodiments, the pharmaceutical composition comprises about 29% water by weight. In some embodiments, the pharmaceutical composition comprises about 30% water by weight.

In some embodiments, the pharmaceutical composition comprises at least about 0.1% by weight of the compound of Formula (I). In some embodiments, the pharmaceutical composition comprises between about 0.1% to about 10% by weight of the compound of Formula (I). In some embodiments, the pharmaceutical composition comprises between about 1% to about 5% by weight of the compound of Formula (I). In some embodiments, the pharmaceutical composition comprises about 0.1% by weight of the compound of Formula (I). In some embodiments, the pharmaceutical composition comprises about 0.2% by weight of the compound of Formula (I). In some embodiments, the pharmaceutical composition comprises about 0.3% by weight of the compound of Formula (I). In some embodiments, the pharmaceutical composition comprises about 0.4% by weight of the compound of Formula (I). In some embodiments, the pharmaceutical composition comprises about 0.5% by weight of the compound of Formula (I). In some embodiments, the pharmaceutical composition comprises about 0.6% by weight of the compound of Formula (I). In some embodiments, the pharmaceutical composition comprises about 0.7% by weight of the compound of Formula (I). In some embodiments, the pharmaceutical composition comprises about 0.8% by weight of the compound of Formula (I). In some embodiments, the pharmaceutical composition comprises about 0.9% by weight of the compound of Formula (I). In some embodiments, the pharmaceutical composition comprises about 1% by weight of the compound of Formula (I). In some embodiments, the pharmaceutical composition comprises about 1.5% by weight of the compound of Formula (I). In some embodiments, the pharmaceutical composition comprises about 2% by weight of the compound of Formula (I). In some embodiments, the pharmaceutical composition comprises about 2.5% by weight of the compound of Formula (I). In some embodiments, the pharmaceutical composition comprises about 3% by weight of the compound of Formula (I). In some embodiments, the pharmaceutical composition comprises about 3.5% by weight of the compound of Formula (I). In some embodiments, the pharmaceutical composition comprises about 4% by weight of the compound of Formula (I). In some embodiments, the pharmaceutical composition comprises about 4.5% by weight of the compound of Formula (I). In some embodiments, the pharmaceutical composition comprises about 5% by weight of the compound of Formula (I). In some embodiments, the pharmaceutical composition comprises about 5.5% by weight of the compound of Formula (I). In some embodiments, the pharmaceutical composition comprises about 6% by weight of the compound of Formula (I). In some embodiments, the pharmaceutical composition comprises about 6.5% by weight of the compound of Formula (I). In some embodiments, the pharmaceutical composition comprises about 7% by weight of the compound of Formula (I). In some embodiments, the pharmaceutical composition comprises about 7.5% by weight of the compound of Formula (I). In some embodiments, the pharmaceutical composition comprises about 8% by weight of the compound of Formula (I). In some embodiments, the pharmaceutical composition comprises about 8.5% by weight of the compound of Formula (I). In some embodiments, the pharmaceutical composition comprises about 9% by weight of the compound of Formula (I). In some embodiments, the pharmaceutical composition comprises about 9.5% by weight of the compound of Formula (I). In some embodiments, the pharmaceutical composition comprises about 10% by weight of the compound of Formula (I).

In some embodiments, the compound of Formula (I) can be present in a composition in an amount of about 10 mg/mL. In some embodiments, the compound of Formula (I) can be present in a composition in an amount of about 20 mg/mL. In some embodiments, the compound of Formula (I) can be present in a composition in an amount of about 25 mg/mL. In some embodiments, the compound of Formula (I) can be present in a composition in an amount of about 50 mg/mL. In some embodiments, the compound of Formula (I) can be present in a composition in an amount of about 75 mg/mL. In some embodiments, the compound of Formula (I) can be present in a composition in an amount of about 100 mg/mL. In some embodiments, the compound of Formula (I) can be present in a composition in an amount of about 150 mg/mL. In some embodiments, the compound of Formula (I) can be present in a composition in an amount of about 200 mg/mL. In some embodiments the compound of Formula (I) can be present in a composition in an amount of about 250 mg/mL. In some embodiments, the compound of Formula (I) can be present in a composition in an amount of about 400 mg/mL.

In some embodiments, the compound of Formula (I) can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

### Pharmaceutical Compositions for Injection

In some embodiments, the disclosure provides a pharmaceutical composition for injection containing a compound of Formula (I) and a pharmaceutical excipient suitable for injection. Components and amounts of agents in the composition are as described herein.

The forms in which a composition of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. In some embodiments, the compositions comprise solvents (e.g., water, alcohols, glycols), solubilizing agents, solvents, and surfactants.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

In some embodiments, the pharmaceutical composition is formulated for parenteral, topical, transdermal, buccal, sublingual, subcutaneous, intramuscular, intravenous, intratumoral, and/or intraperitoneal administration. In some embodiments, the pharmaceutical composition is formulated for parenteral administration. In some embodiments, the pharmaceutical composition is formulated for injection. In some embodiments, the pharmaceutical composition is formulated for intratumoral injection. In some embodiments, the pharmaceutical composition is formulated as an injection, a patch, a cream, a gel, or an ointment.

### Kits

The invention also provides kits. The kits may include a pharmaceutical composition comprising a compound of Formula (I) and one or more additional agents in suitable packaging with written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound of the present invention and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### EXAMPLES

### Example 1: Clinical Trial

The clinical trial described herein was a double-blind, randomized, placebo-controlled phase 2b clinical trial for the evaluation of safety and efficacy of Compound 1 (5-(3,6-dibromo-9H-carbazol-9-yl)-N,N,N-trimethylpentan-1-aminium, provided as a chloride salt, also designated Compound 1A), in subjects having Dercum's disease (DD) lipomas.

Study Objectives. The primary objective was to evaluate the average percent reduction in lipoma height on day 84 after injection vs. baseline in the treatment group compared to the placebo group. Efficacy was determined by ultrasound assessment of the lipoma/nodule dimensions after treatment vs. baseline. The key secondary objective was assessment of lipoma/nodule associated pain using the Comparative Pain Scale. Safety was assessed by frequency of adverse events and by change-from-baseline values for vital signs, clinical laboratory and ECG.

Study Design. This was a double blind, multi-center, randomized, placebo-controlled clinical trial in DD subjects having lipomas. A total of 38 DD patients were enrolled into the study. Patients were randomized in approximately a 1:1 ratio into either the intervention or the placebo arm. Twenty (20) subjects, having a total of 125 lipomas, were treated with Compound 1. Eighteen (18) subjects, having a total of 110 lipomas, were treated with placebo. Once the study ended and codes were opened, 84 days after dosing.

Subjects received their study treatment on a single occasion in multiple injections. Dosing was calculated according to lipoma size (diameter) as determined by ultrasound.

Subjects were dosed in accordance with Table 1. Injections were perpendicular (90°) to injected skin surface and spread randomly, at least 1 cm apart.

TABLE 1

| Number of Subjects - | 20/18 | | | | |
|---|---|---|---|---|---|
| Active/Placebo | | | | | |
| Lipoma/Nodule size - | 1-1.9 | 2-3.9 | 4-5.9 | 6-7.9 | 8-10 |
| diameter (cm) | | | | | |
| Total Dose of | 10 | 20 | 40 | 50 | 60 |
| Compound 1 (mg) | | | | | |
| Number of Injections | 2 | 4 | 8 | 10 | 12 |

At least 4 lipomas/nodules, preferably 6, and no more than 8, were injected per subject. Compound 1 dose per injection site was 5 mg, and a distance of 1-3 cm between injection points was maintained, to allow good distribution of Compound 1 within lipoma fat. A maximal dose of up to 240 mg/subject (a total of 48 injections of 5 mg/injection) was allowed in this study and the highest actual tested dose was 200 mg/subject (40 injections into 4-8 lipomas). A total of 20 patients were treated with Compound 1 and a total of 18 patients were treated with placebo.

Primary endpoint was the average percent reduction in lipoma/noduleheight on day 84 after injection vs baseline-evaluated by ultrasound in the Compound 1 treatment group compared to the placebo group. Key secondary endpoint was reduction in local pain score per lipoma/nodule on Day 84 vs. baseline as measured by the Comparative Pain Scale. Comparative pain scale ranked the pain from a score from 0 to 10. A score of 0 indicated no pain, a score of 1-3 indicated minor pain, a score of 4-6 indicated moderate pain and a score of 7-10 indicated severe pain. Pain was compared between the intervention (Compound 1) and placebo groups on 2 levels—(1) on the patient level and (2) by lipomas, i.e. comparison between all injected lipomas before and after injection.

Study Drug. Compound 1 was administered as a ready to use liquid for injection into subcutaneous fat, supplied in a 1 vial kit. Each vial contains 250 mg Compound 1/5 mL (50 mg/ml) solution Compound 1 in a vehicle comprised of water, propylene glycol and surfactant. The formulation is described in Table 2.

TABLE 2

| Component | Function | Concentration (mg/ml) |
|---|---|---|
| Compound 1 | Active Ingredient | 50 |
| Tween-80 | Surfactant | 100 |
| Propylene glycol | Solvent | 570 |
| Benzyl Alcohol | Solubilizing agent | 30 |
| Water | Solvent | 250 |

Placebo was a solution comprised of vehicle only. Each injection of Compound 1 and placebo contained 0.1 ml. The vehicle was a ready-to-use liquid for injection into subcutaneous fat, supplied in a 1-vial kit. One vial contained 5 mL and had similar composition as the Compound 1 drug product, without the active substance. The components are: Tween-80, propylene glycol, benzyl alcohol, and water. The vehicle was manufactured and packed by Nextar (Israel) and complies with cGMP requirements.

### Statistical Methods

All measured variables and derived parameters were listed individually and, if appropriate, tabulated by descriptive statistics. For categorical variables summary tables are provided giving sample size, absolute and relative frequency and 95% CI (Confidence Interval) for proportions by study group. For continuous variables summary tables are provided giving sample size, arithmetic mean, standard deviation, coefficient of variation (CV %), median, minimum and maximum and 95% CI (Confidence Interval) for means of variables by study group. All tests were two-tailed, and a p value of 5% or less will be considered statistically significant. Data was analyzed using the SAS® version 9.4 (SAS Institute, Cary North Carolina).

Analysis per patient (based on 4-8 lipomas/nodules per patient) The two-sample T-test or Non-parametric Wilcoxon-Mann-Whitney Rank sum test for independent samples (as is appropriate) was applied for testing the statistical significance of the difference between study groups in percent reduction of lipoma/nodule height at day 84 after injection vs. baseline. Analysis of covariance (ANCOVA) model will be applied in order to identify covariate parameters suspected as related to reduction in lipoma/nodule height and in order to test the differences in percent reduction of lipoma/nodules height between the treatment groups adjusted to the above covariates.

Analysis per lipoma/nodule (based on 4-8 lipomas/nodules per patient. The MMRM model (Mixed-effect model for repeated measures) was applied for analyzing the difference between the groups in percent reduction of lipoma/nodule height from baseline to day 84, and adjusted for the above covariates as appropriate.

### Results

The effect of Compound 1 on lipomas of Dercum's Disease was assessed based on the following: (1) Change in lipoma height and other dimensions based on ultrasound measurements of each injected lipoma at the baseline visit and post-treatment follow-up visits, Day 28, 56 and 84 following injections; and (2) Change in lipoma pain based on Comparative Pain Scale for each injected lipoma at the baseline visit and post-treatment follow-up visits, Day 28, 56 and 84 following injections.

### Change in Lipoma Pain

Pain of each injected lipoma was assessed by the blinded physician at baseline visit prior to injection and at each of the follow-up visits by the Comparative Pain Scale as described herein.

(i) Change in lipoma Pain Per Lipoma

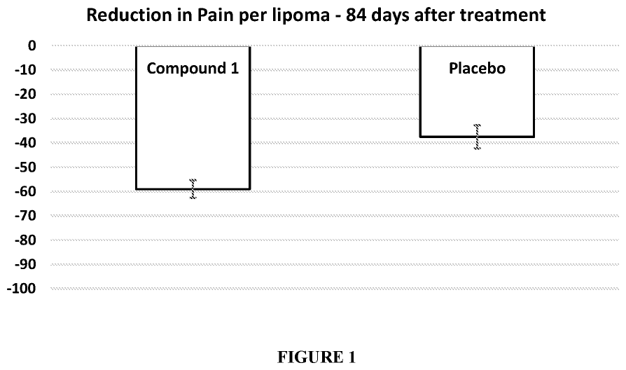

A total of 125 lipomas were assessed for pain in baseline in the Compound 1-treatment group and 108 lipomas in the placebo group. Table 3 summarizes the average all-lipomas pain by treatment group and study visit. The change in lipoma pain from baseline according to analysis of all lipomas (in percentages) is displayed in Table 4. FIG. 1 depicts percent reduction of pain per lipoma 84 days after treatment.

The results show a significant reduction in pain per lipoma in the Compound 1 treatment group vs. the placebo. The all-lipoma analysis demonstrated an average pain reduction of 59.06%±40.41 in the Compound 1 treated group vs. 37.50%±49.69 in the placebo group. The statistical significance tested by Mixed model with repeated measure generated a p value of 0.079. Calculation of statistical difference using t-test generated a p value of 0.0004.

TABLE 3
Pain Score: Average per lipomas analysis

| Pain score: All lipomas | Compound 1 | | | Placebo | | |
|---|---|---|---|---|---|---|
| Visit | N | Mean | Std | N | Mean | Std |
| Visit 2 - Baseline/Treatment (Day 0) | 125 | 4.86 | 2.03 | 108 | 4.82 | 2.25 |
| Visit 5 - Day 28 post treatment | 125 | 2.72 | 2.14 | 109 | 3.17 | 2.74 |
| Visit 6 - Day 56 post treatment | 119 | 2.38 | 2.28 | 109 | 3.02 | 2.84 |
| Visit 7 - Day 84 post treatment | 125 | 1.86 | 2.00 | 109 | 2.94 | 2.60 |

TABLE 4
Change from baseline in Pain score: Average per lipomas analysis

| Change from baseline in Pain Score: | | Compound 1 | | | Placebo | | |
|---|---|---|---|---|---|---|---|
| All lipomas | | N | Mean | Std | N | Mean | Std |
| Visit 5 - Day | Relative | 125 | −41.55 | 42.26 | 108 | −31.79 | 47.73 |
| 28 post | Change from | | | | | | |
| treatment | baseline (%) | | | | | | |
| Visit 6 - Day | Relative | 119 | −46.74 | 56.82 | 108 | −37.11 | 46.86 |
| 56 post | Change from | | | | | | |
| treatment | baseline (%) | | | | | | |
| Visit 7 - Day | Relative | 125 | −59.06 | 40.41 | 108 | −37.50 | 49.69 |
| 84 post | Change from | | | | | | |
| treatment | baseline (%) | | | | | | |

### (ii) Change in Lipoma Pain Per Patient

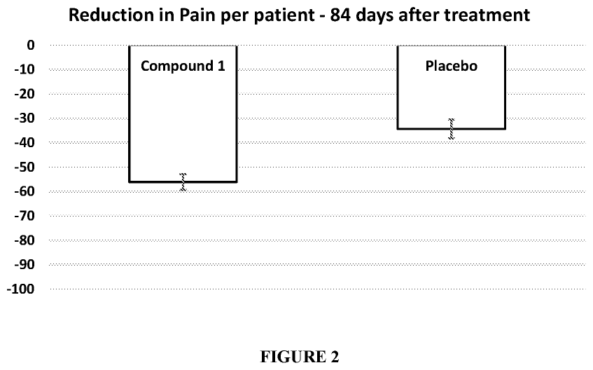

Table 5 displays the average by-patient pain by treatment group and study visit. The change in lipoma pain from baseline according to analysis by patient (in percentages) is displayed in Table 6. The by-patient analysis demonstrated an average pain reduction of 56.37%±35.72 in the treated subjects vs. an average pain reduction of 36.82%±37.20 in the placebo-treated subjects. (P=0.98) FIG. 2 depicts percent reduction of pain per patient 84 days after treatment.

TABLE 5

| Pain Score: Average per patient | Compound 1 | | | Placebo | | |
|---|---|---|---|---|---|---|
| Visit | N | Mean | Std | N | Mean | Std |
| Visit 2 - Baseline/Treatment | 20 | 4.89 | 1.78 | 18 | 4.74 | 1.99 |
| (Day 0) | | | | | | |
| Visit 5 - Day 28 post treatment | 20 | 2.82 | 1.95 | 18 | 3.03 | 2.55 |
| Visit 6 - Day 56 post treatment | 19 | 2.56 | 2.24 | 18 | 2.89 | 2.55 |
| Visit 7 - Day 84 post treatment | 20 | 2.03 | 1.87 | 18 | 2.91 | 2.21 |

TABLE 6
Change from baseline in Pain score: Average per patient

| Change from baseline in Pain | | Compound 1 | | | Placebo | | |
|---|---|---|---|---|---|---|---|
| Score: Average per patient | | N | Mean | Std | N | Mean | Std |
| Visit 5 - Day | Relative | 20 | −41.76 | 29.70 | 18 | −34.40 | 44.49 |
| 28 post | Change from | | | | | | |
| treatment | baseline (%) | | | | | | |
| Visit 6 - Day | Relative | 19 | −43.53 | 50.39 | 18 | −40.17 | 35.95 |
| 56 post | Change from | | | | | | |
| treatment | baseline (%) | | | | | | |
| Visit 7 - Day | Relative | 20 | −56.37 | 35.72 | 18 | −36.82 | 37.20 |
| 84 post | Change from | | | | | | |
| treatment | baseline (%) | | | | | | |

### Conclusions

Although reduction is lipoma height was not significant, Compound 1 demonstrated a statistically significant reduction in pain in the by-lipoma analysis, and a trend for reduction of pain in the per-patient analysis, as compared to the placebo group. Without wishing to be bound by any theory or mechanism of action, the efficacy of Compound 1 treatment for pain reduction may be related to the fact that fat tissue was lysed in the injected lipoma and might cause the lipomas to be less dense which leads to less pressure on the nerves by the lipomas following treatment.

The compound was generally well tolerated There were no reported serious adverse events, and no clinically significant changes in blood parameters. Adverse events were mostly injection site edema pain and pruritus.

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

What is claimed is:

1. A method of treating lipoma pain, the method comprising the step of administering to a subject in need thereof a therapeutically-effective amount of a pharmaceutical composition in unit dosage form, the pharmaceutical compositing comprising a compound of formula (I): 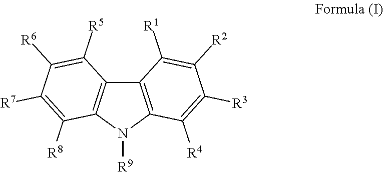
or a pharmaceutically-acceptable salt thereof, wherein:
each of R¹, R², R³, R⁴, R⁵, R⁶, R⁷, and R⁸ is independently H, halogen, —CN, —NO², —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(—O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(—O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

2. The method of claim 1, wherein treating lipoma pain comprises reducing or alleviating pain associated with a lipoma.

3. The method of claim 2, wherein pain is reduced by at least 30% relative to pre-treatment.

4. The method of claim 2, wherein pain is reduced by at least 50% relative to pre-treatment.

5. The method of any one of the preceding claims, wherein pain is alleviated for at least about 60 days.

6. The method of any one of the preceding claims, wherein pain is alleviated for at least about 60 days after a single administration of compound (I).

7. The method of any one of the preceding claims, wherein the subject has Dercum's Disease.

8. The method of any one of the preceding claims, wherein R⁹ is C₁-C₉ alkyl substituted with at least one quaternary ammonium group.

9. The method of claim 8, wherein the at least one ammonium group is a group of Formula (V): 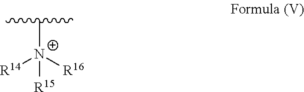
wherein each of R¹⁴, R¹⁵, and R¹⁶ is independently C₁₋₉alkyl, C₂₋₉alkenyl, or C₂₋₉alkynyl.

10. The method of claim 9, wherein the at least one ammonium group is a group of Formula (V′): 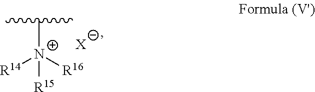
wherein X is a negatively charged ion.

11. The method of claim 10, wherein X is Cl.

12. The method of any one of claims 9-11, wherein each of R¹⁴, R¹⁵, and R¹⁶ is independently methyl

13. The method of any one of the preceding claims, wherein at least one of R¹, R², R³, and R⁴ is halogen.

14. The method of any one of the preceding claims, wherein at least one of R⁵, R⁶, R⁷, and R⁸ is halogen.

15. The method of any one of the preceding claims, wherein at least one of R¹, R², R³, and R⁴ is halogen and at least one of R⁵, R⁶, R⁷, and R⁸ is halogen.

16. The method of any one of claims 13 to 15, wherein the halogen is bromo.

17. The method of any one of the preceding claims, wherein at least one of R¹, R², R³, and R⁴ is OH.

18. The method of any one of the preceding claims, wherein at least one of R⁵, R⁶, R⁷, and R⁸ is OH.

19. The method of any one of the preceding claims, wherein at least one of R¹, R², R³, and R⁴ is nitro and at least one of R⁵, R⁶, R⁷, and R⁸ is nitro.

20. The method of any one of the preceding claims, wherein the compound of Formula (I) is:
3-(3,6-dibromo-9H-carbazol-9-yl)-N,N,N-trimethylpropan-1-aminium;
5-(9H-carbazol-9-yl)-N,N,N-trimethylpentan-1-aminium;
5-(2-hydroxy-9H-carbazol-9-yl)-N,N,N-trimethylpentan-1-aminium; or
5-(3,6-dibromo-9H-carbazol-9-yl)-N,N,N-trimethylpentan-1-aminium.

21. The method of any one of the preceding claims, wherein the compound of Formula (I) is represented by the structure of formula (1)

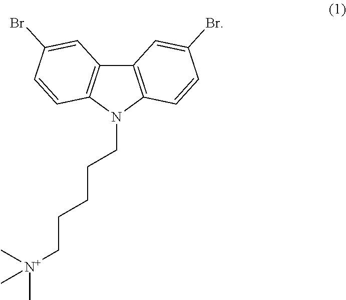 [SMILES: C[N+](C)(C)CCCCCn1c2ccc(Br)cc2c2cc(Br)ccc21]

22. The method of any one of the preceding claims, wherein the compound of Formula (I) is 5-(3,6-dibromo-9H-carbazol-9-yl)-N,N,N-trimethylpentan-1-aminium chloride.

23. The method of any one of the preceding claims, wherein the pharmaceutical composition further comprises at least one pharmaceutically-acceptable excipient.

24. The method of claim 23, wherein the excipient is a surfactant.

25. The method of claim 24, wherein the surfactant is Tween-80.

26. The method of claim 23, wherein the excipient is a solubilizing agent.

27. The method of claim 26, wherein the solubilizing agent is benzyl alcohol.

28. The method of claim 23, wherein the excipient is a solvent.

29. The method of claim 28, wherein the solvent is propylene glycol.

30. The method of claim 28, wherein the solvent is water.

31. The method of any one of the preceding claims, wherein the pharmaceutical composition comprises less than about 50% water by weight.

32. The method of claim 31, wherein the pharmaceutical composition comprises less than about 30% water by weight.

33. The method of claim 31, wherein the pharmaceutical composition comprises less than about 10% water by weight.

34. The method of claim 31, wherein the pharmaceutical composition comprises from about 10% to about 30% water by weight.

35. The method of any one of the preceding claims, wherein the pharmaceutical composition comprises at least about 0.1% by weight of the compound of Formula (I).

36. The method of claim 35, wherein the pharmaceutical composition comprises from about 0.1% to about 10% by weight of the compound of Formula (I).

37. The method of claim 35, wherein the pharmaceutical composition comprises from about 1% to about 5% by weight of the compound of Formula (I).

38. The method of any one of the preceding claims, wherein the pharmaceutical composition comprises from 1 to 100 mg compound of formula (I) per mL.

39. The method of claim 38, wherein the pharmaceutical composition comprises 50 mg of the compound of formula (I) per mL.

40. The method of any one of the preceding claims, wherein the pharmaceutical composition is formulated in a liquid dosage form.

41. The method of any one of the preceding claims, wherein the pharmaceutical composition is administered in a single injection.

42. The method of any one of the preceding claims, wherein the pharmaceutical composition is administered in multiple injections.

43. The method of any one of the preceding claims, wherein the pharmaceutical composition is administered parenterally.

44. The method of any one of the preceding claims, wherein the pharmaceutical composition is administered subcutaneously.

45. The method of claim 44, wherein the pharmaceutical composition is subcutaneously injected directly into a lipoma.

46. The method of claim 45, wherein the pharmaceutical composition is subcutaneously injected directly into a lipoma at a dosage of from about 1 mg to about 10 mg per cm of lipoma.

47. The method of claim 45, wherein the pharmaceutical composition is subcutaneously injected directly into a lipoma at a dosage of from about 5 mg to about 10 mg per cm of lipoma.

48. The method of any one of the preceding claims, wherein the pharmaceutical composition further comprises at least one additional active agent.

49. A pharmaceutical composition in unit dosage form, compositing comprising a compound of formula (I): 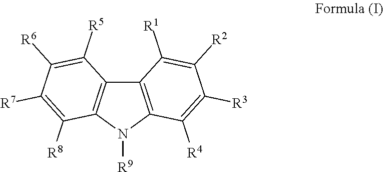
or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl,
for use in the treatment of lipoma pain.

50. The pharmaceutical composition for use according to claim 49, wherein treating lipoma pain comprises reducing or alleviating pain associated with a lipoma.

51. The pharmaceutical composition for use according to claim 49, wherein pain is reduced by at least 30% relative to pre-treatment.

52. The pharmaceutical composition for use according to claim 49, wherein pain is reduced by at least 50% relative to pre-treatment.

53. The pharmaceutical composition for use of any one of claims 49-51, wherein pain is alleviated for at least about 60 days.

54. The pharmaceutical composition for use of any one of claims 49-52, wherein pain is alleviated for at least about 60 days after a single administration of compound (I).

55. The pharmaceutical composition for use according to any one of claims 49-54, wherein the subject has Dercum's Disease.

56. The pharmaceutical composition for use according to any one of claims 49-55, wherein R⁹ is C₁-C₉ alkyl substituted with at least one quaternary ammonium group.

57. The pharmaceutical composition for use according to claim 56, wherein the at least one ammonium group is a group of Formula (V): 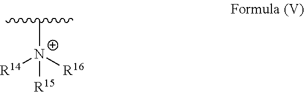
wherein each of R¹⁴, R¹⁵, and R¹⁶ is independently C₁₋₉alkyl, C₂₋₉alkenyl, or C₂₋₉alkynyl.

58. The pharmaceutical composition for use according to claim 57, wherein the at least one ammonium group is a group of Formula (V′): 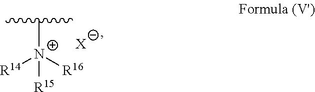
wherein X is a negatively charged ion.

59. The pharmaceutical composition for use of claim 58, wherein X is Cl.

60. The pharmaceutical composition for use according to claim 57, wherein each of R¹⁴, R¹⁵, and R¹⁶ is independently methyl.

61. The pharmaceutical composition for use according to any one of claims 49-60, wherein at least one of R¹, R², R³, and R⁴ is halogen.

62. The pharmaceutical composition for use according to any one of claims 49-60, wherein at least one of R⁵, R⁶, R⁷, and R⁸ is halogen.

63. The pharmaceutical composition for use according to any one of claims 49-60, wherein at least one of R¹, R², R³, and R⁴ is halogen and at least one of R⁵, R⁶, R⁷, and R⁸ is halogen.

64. The pharmaceutical composition for use according to any one of claims 61 to 63, wherein the halogen is bromo.

65. The pharmaceutical composition for use according to any one of claims 49-60, wherein at least one of R¹, R², R³, and R⁴ is OH.

66. The pharmaceutical composition for use according to any one of claims 49-60, wherein at least one of R⁵, R⁶, R⁷, and R⁸ is OH.

67. The pharmaceutical composition for use according to any one of claims 49-60, wherein at least one of R¹, R², R³, and R⁴ is nitro and at least one of R⁵, R⁶, R⁷, and R⁸ is nitro.

68. The pharmaceutical composition for use according to any one of claims 49-67, wherein the compound of Formula (I) is:
3-(3,6-dibromo-9H-carbazol-9-yl)-N,N,N-trimethylpropan-1-aminium;
5-(9H-carbazol-9-yl)-N,N,N-trimethylpentan-1-aminium;
5-(2-hydroxy-9H-carbazol-9-yl)-N,N,N-trimethylpentan-1-aminium; or
5-(3,6-dibromo-9H-carbazol-9-yl)-N,N,N-trimethylpentan-1-aminium.

69. The pharmaceutical composition for use according to any one of claims 49-68, wherein the compound of Formula (I) is represented by the structure of formula (1)

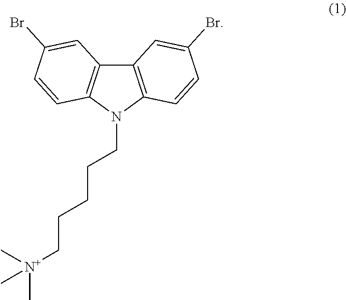 [SMILES: C[N+](C)(C)CCCCCn1c2ccc(Br)cc2c2cc(Br)ccc21]

70. The pharmaceutical composition for use according to any one of claims 49-69, wherein the compound of Formula (I) is 5-(3,6-dibromo-9H-carbazol-9-yl)-N,N,N-trimethylpentan-1-aminium chloride.

71. The pharmaceutical composition for use according to any one of claims 49-69, wherein the pharmaceutical composition further comprises at least one pharmaceutically-acceptable excipient.

72. The pharmaceutical composition for use according to claim 70, wherein the excipient is a surfactant.

73. The pharmaceutical composition for use according to claim 72, wherein the surfactant is Tween-80.

74. The pharmaceutical composition for use according to claim 70, wherein the excipient is a solubilizing agent.

75. The pharmaceutical composition for use according to claim 74, wherein the solubilizing agent is benzyl alcohol.

76. The pharmaceutical composition for use according to claim 74, wherein the excipient is a solvent.

77. The pharmaceutical composition for use according to claim 76, wherein the solvent is propylene glycol.

78. The pharmaceutical composition for use according to claim 76, wherein the solvent is water.

79. The pharmaceutical composition for use according to any one of claims 49-78, wherein the pharmaceutical composition comprises less than about 50% water by weight.

80. The pharmaceutical composition for use according to any one of claims 49-79, wherein the pharmaceutical composition comprises less than about 30% water by weight.

81. The pharmaceutical composition for use according to any one of claims 49-80, wherein the pharmaceutical composition comprises less than about 10% water by weight.

82. The pharmaceutical composition for use according to any one of claims 49-81, wherein the pharmaceutical composition comprises from about 10% to about 30% water by weight.

83. The pharmaceutical composition for use according to any one of claims 49-82, wherein the pharmaceutical composition comprises at least about 0.1% by weight of the compound of Formula (I).

84. The pharmaceutical composition for use according to any one of claims 49-83, wherein the pharmaceutical composition comprises from about 0.1% to about 10% by weight of the compound of Formula (I).

85. The pharmaceutical composition for use according to any one of claims 49-84, wherein the pharmaceutical composition comprises from about 1% to about 5% by weight of the compound of Formula (I).

86. The pharmaceutical composition for use according to any one of claims 49-85, wherein the pharmaceutical composition comprises from 1 to 100 mg compound of formula (I) per mL.

87. The pharmaceutical composition for use according to claim 86, wherein the pharmaceutical composition comprises 50 mg of the compound of formula (I) per mL.

88. The pharmaceutical composition for use according to any one of claims 49-87, wherein the pharmaceutical composition is formulated in a liquid dosage form.

89. The pharmaceutical composition for use according to any one of claims 49-88, wherein the pharmaceutical composition is formulated for a single injection.

90. The pharmaceutical composition for use according to any one of claims 49-88, wherein the pharmaceutical composition is formulated for multiple injections.

91. The pharmaceutical composition for use according to any one of claims 49-90, wherein the pharmaceutical composition is formulated for parenteral administration.

92. The pharmaceutical composition for use according to any one of claims 49-90, wherein the pharmaceutical composition is formulated for subcutaneous administration

93. The pharmaceutical composition for use according to claim 92, wherein the pharmaceutical composition is subcutaneously injected directly into a lipoma.

94. The pharmaceutical composition for use according to claim 93, wherein the pharmaceutical composition is subcutaneously injected directly into a lipoma at a dosage of from about 1 mg to about 10 mg per cm of lipoma.

95. The pharmaceutical composition for use according to claim 93, wherein the pharmaceutical composition is subcutaneously injected directly into a lipoma at a dosage of from about 5 mg to about 10 mg per cm of lipoma.

96. The pharmaceutical composition for use according to any one of claims 44-95, wherein the pharmaceutical composition further comprises at least one additional active agent.

97. A kit comprising a pharmaceutical composition according to any one of claims 44-96, means for administration of the pharmaceutical composition, and instructions for use thereof.

98. The kit of claim 97, further comprising at least one additional therapeutic agent.
